(19) United States
(12) Patent Application Publication
(10) Pub. No.: US 2010/0305448 A1
(43) Pub. Date: Dec. 2, 2010

(54) APPARATUS AND METHOD FOR INDICATING ULTRASOUND PROBE ORIENTATION AND ACTIVATION STATUS

(76) Inventors: Anne Cecile Dagonneau, Grasse (FR); Jean Marc Hebrard, Sophia Antipolis (FR); Trond Kleveland, Horten (NO); Svein Bergstoel, Sandefjord (NO); Kjell Kristoffersen, Oslo (NO)

Correspondence Address: DEAN D. SMALL;THE SMALL PATENT LAW GROUP LLP, 225 S. MERAMEC, STE. 725T, ST. LOUIS, MO, 63105, US

(21) Appl. No.: 12/471,721

(22) Filed: May 26, 2009

(51) Int. Cl. A61B 8/14

(52) U.S. Cl. 600/459

(57) ABSTRACT

An ultrasound probe has a probe casing and a light emitting diode (LED). The probe casing has an inner surface and an outer surface. The LED is mounted and contained entirely within the probe casing and the LED is configured to be illuminated when the probe is active.

### BACKGROUND OF THE INVENTION

The subject matter disclosed herein relates generally to ultrasound probes, and more particularly to apparatus and methods for indicating an orientation and activation status of a probe.

Some ultrasound probes are provided with an LED that protrudes through a hole in the casing. In some cases the LED may also be encapsulated in a plastic cap. The LED is illuminated when the probe is activated. Therefore, the operator of the ultrasound system knows which probe is currently activated. The LED may also be used by the operator to identify the orientation of the probe when scanning a patient. However, because the LED passes through the casing asperities and/or grooves are formed on the outer surface of the casing. The grooves and roughness in the outer surface are very difficult to clean, which can lead to contamination of the casing area around the LED.

Extensive cleaning of the LED and surrounding area may be time consuming for the operator as well as detrimental to the casing and the lens of the probe, which is typically located near the LED. Abrasions of the lens material resulting from the cleaning procedures may degrade the quality of the acquired ultrasound image.

### BRIEF DESCRIPTION OF THE INVENTION

In one embodiment, an ultrasound probe comprises a probe casing and a light emitting diode (LED). The probe casing comprises an inner surface and an outer surface. The LED is mounted and contained entirely within the probe casing and the LED is configured to be illuminated when the probe is active.

In another embodiment, a method for indicating whether an ultrasound probe is active comprises locating an LED beneath a probe casing. The probe casing is configured to enclose at least the LED and an array of transducer elements. Power is provided to the LED when the probe is selected, and the LED is configured to provide a level of luminescence that is visible outside the probe casing.

In yet another embodiment, an ultrasound probe comprises a probe casing comprising an inner surface and an outer surface. The probe casing has a thickness. An LED is mounted within the probe casing and is positioned to indicate an orientation of the probe. The thickness of the probe casing in a region proximate the LED is thinner than in areas of the probe casing located away from the LED. The LED is configured to have a luminescence that is visible outside the outer surface when the LED is activated.

### BRIEF DESCRIPTION OF THE DRAWINGS

FIG. 1 illustrates an ultrasound system formed in accordance with an embodiment of the present invention.

FIG. 2 illustrates a 3D-capable miniaturized ultrasound system formed in accordance with an embodiment of the present invention.

FIG. 3 illustrates a mobile ultrasound imaging system formed in accordance with an embodiment of the present invention.

FIG. 4 illustrates a hand carried or pocket-sized ultrasound imaging system formed in accordance with an embodiment of the present invention.

FIG. 5 illustrates an exemplary cross-sectional view of the probe of FIG. 1 having a light emitting diode (LED) located beneath an outer cover or casing in accordance with an embodiment of the present invention.

FIG. 6 illustrates a cut-away view of a casing and LED formed in accordance with an embodiment of the present invention.

FIG. 7 illustrates a cup-shaped cavity formed in a casing in accordance with an embodiment of the present invention.

FIG. 8 illustrates a cut-away view of a casing formed in accordance with an embodiment of the present invention.

FIG. 9 illustrates another cut-away view that shows a cup-shaped cavity formed in a casing in accordance with an embodiment of the present invention.

FIG. 10 illustrates another embodiment wherein the LED is located beneath the casing of the probe of FIG. 1 in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

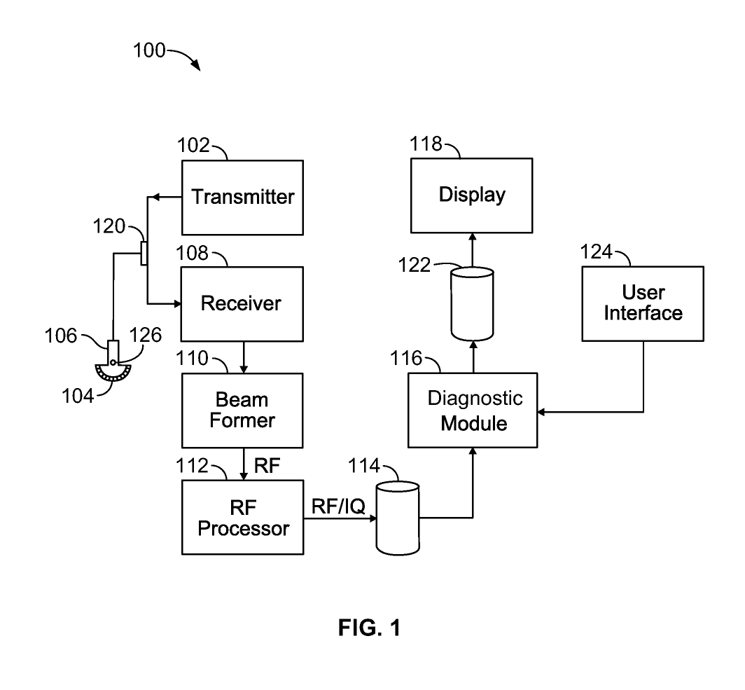

FIG. 1 illustrates an ultrasound system 100 including a transmitter 102 that drives an array of elements 104 (e.g., piezoelectric elements) within a probe 106 to emit pulsed ultrasonic signals into a body. The elements 104 may be arranged, for example, in one or two dimensions. A variety of geometries may be used, and the probe 106 may be capable of acquiring one, two, three and/or four dimensional image data. The system 100 may have a probe port 120 for receiving the probe 106 or the probe 106 may be hardwired to the system 100. A light emitting diode (LED) 126 may be provided, for example, beneath an outer casing of the probe 106. When activated, illumination from the LED 126 may be seen from outside the outer casing of the probe 106.

The ultrasonic signals are back-scattered from structures in the body, like fatty tissue or muscular tissue, to produce echoes that return to the elements 104. The echoes are received by a receiver 108. The received echoes are passed through a beamformer 110 that performs beamforming and outputs a radiofrequency (RF) signal. The RF signal then passes through an RF processor 112. Alternatively, the RF processor 112 may include a complex demodulator (not shown) that demodulates the RF signal to form in-phase and quadrature (IQ) data pairs representative of the echo signals. The RF or IQ signal data may then be routed directly to a memory 114 for storage.

The ultrasound system 100 also includes a processor module 116 to process the acquired ultrasound information (e.g., RF signal data or IQ data pairs) and prepare frames of ultrasound information for display on display 118. The processor module 116 is adapted to perform one or more processing operations according to a plurality of selectable ultrasound modalities on the acquired ultrasound information. Acquired ultrasound information may be processed and displayed in real-time during a scanning session as the echo signals are received. Additionally or alternatively, the ultrasound information may be stored temporarily in memory 114 or memory 122 during a scanning session and then processed and displayed in an off-line operation.

A user interface 124 may be used to input data to the system 100, adjust settings, and control the operation of the processor module 116. The user interface 124 may have a keyboard, trackball and/or mouse, and a number of knobs, switches or other input devices such as a touchscreen. The display 118 includes one or more monitors that present patient information, including diagnostic ultrasound images to the user for diagnosis and analysis. One or both of memory 114 and memory 122 may store two-dimensional (2D) and/or three-dimensional (3D) datasets of the ultrasound data, where such datasets are accessed to present 2D and/or 3D images. Multiple consecutive 3D datasets may also be acquired and stored over time, such as to provide real-time 3D or four-dimensional (4D) display. The images may be modified and the display settings of the display 118 also manually adjusted using the user interface 124.

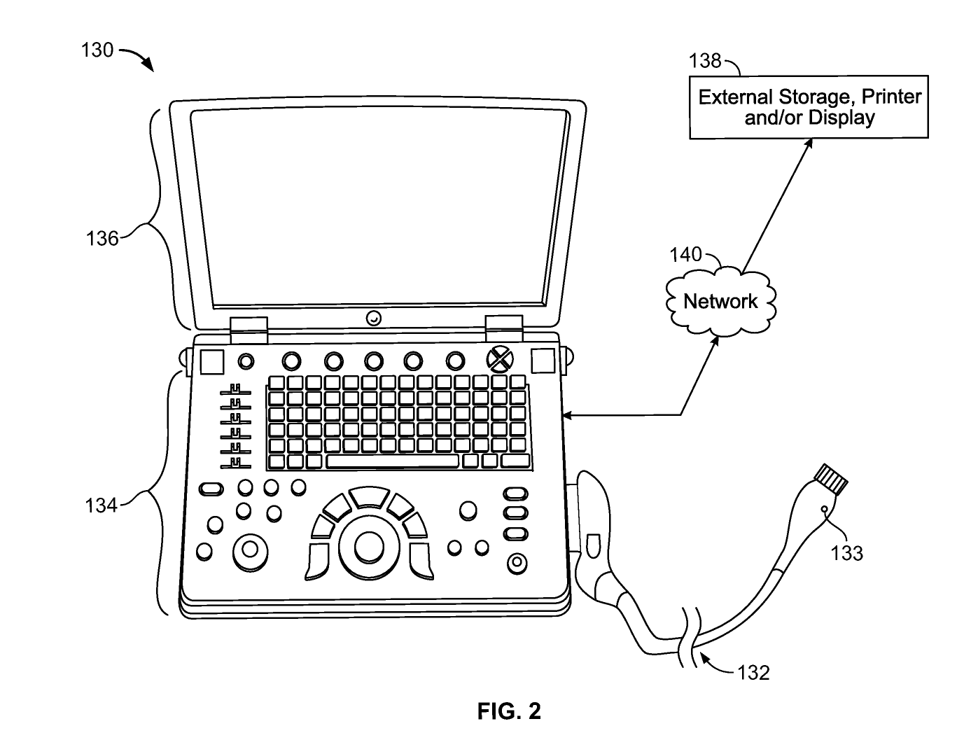

FIG. 2 illustrates a 3D-capable miniaturized ultrasound system 130 having a probe 132 that has an LED 133 located beneath an outer surface or casing of the probe 132. The probe 132 may be configured to acquire 3D ultrasonic data. For example, the probe 132 may have a 2D array of transducer elements 104. A user interface 134 (that may also include an integrated display 136) is provided to receive commands from an operator.

As used herein, “miniaturized” means that the ultrasound system 130 is a handheld or hand-carried device or is configured to be carried in a person's hand, pocket, briefcase-sized case, or backpack. For example, the ultrasound system 130 may be a hand-carried device having a size of a typical laptop computer, for instance, having dimensions of approximately 2.5 inches in depth, approximately 14 inches in width, and approximately 12 inches in height. The ultrasound system 130 may weigh about ten pounds, and thus is easily portable by the operator. The integrated display 136 (e.g., an internal display) is also provided and is configured to display a medical image.

The ultrasonic data may be sent to an external device 138 via a wired or wireless network 140 (or direct connection, for example, via a serial or parallel cable or USB port). In some embodiments, external device 138 may be a computer or a workstation having a display. Alternatively, external device 138 may be a separate external display or a printer capable of receiving image data from the hand carried ultrasound system 130 and of displaying or printing images that may have greater resolution than the integrated display 136. It should be noted that the various embodiments may be implemented in connection with a miniaturized ultrasound system having different dimensions, weights, and power consumption.

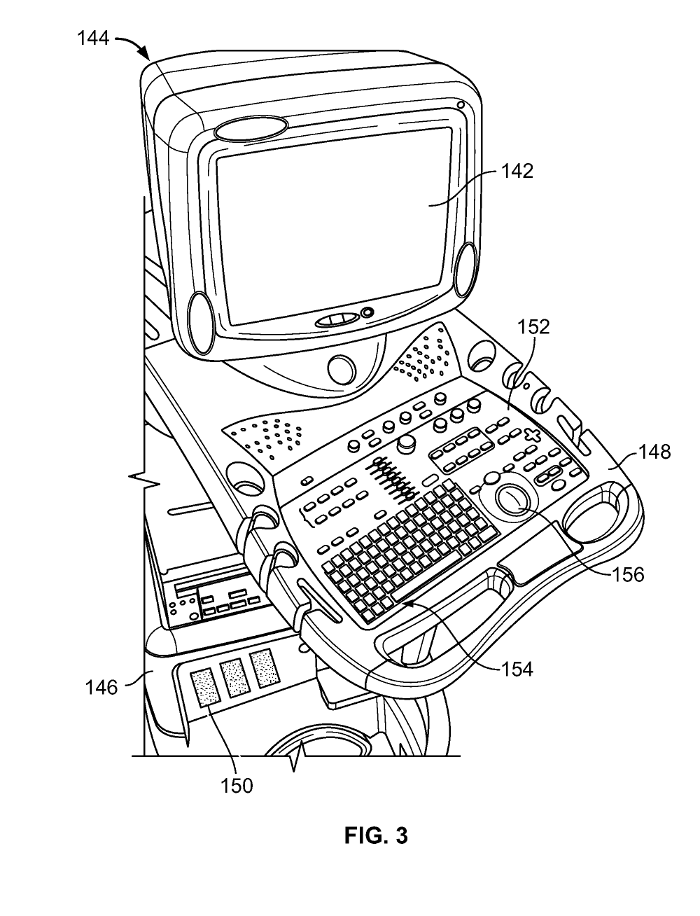

FIG. 3 illustrates a mobile ultrasound imaging system 144 provided on a movable base 146. The ultrasound imaging system 144 may also be referred to as a cart-based system. A display 142 and user interface 148 are provided and it should be understood that the display 142 may be separate or separable from the user interface 148. The system 144 has at least one probe port 150 for accepting probes (not shown) that may have an LED mounted beneath an outer casing as discussed herein.

The user interface 148 may optionally be a touchscreen, allowing the operator to select options by touching displayed graphics, icons, and the like. The user interface 148 also includes control buttons 152 that may be used to control the ultrasound imaging system 144 as desired or needed, and/or as typically provided. The user interface 148 provides multiple interface options that the user may physically manipulate to interact with ultrasound data and other data that may be displayed, as well as to input information and set and change scanning parameters. The interface options may be used for specific inputs, programmable inputs, contextual inputs, and the like. For example, a keyboard 154 and track ball 156 may be provided.

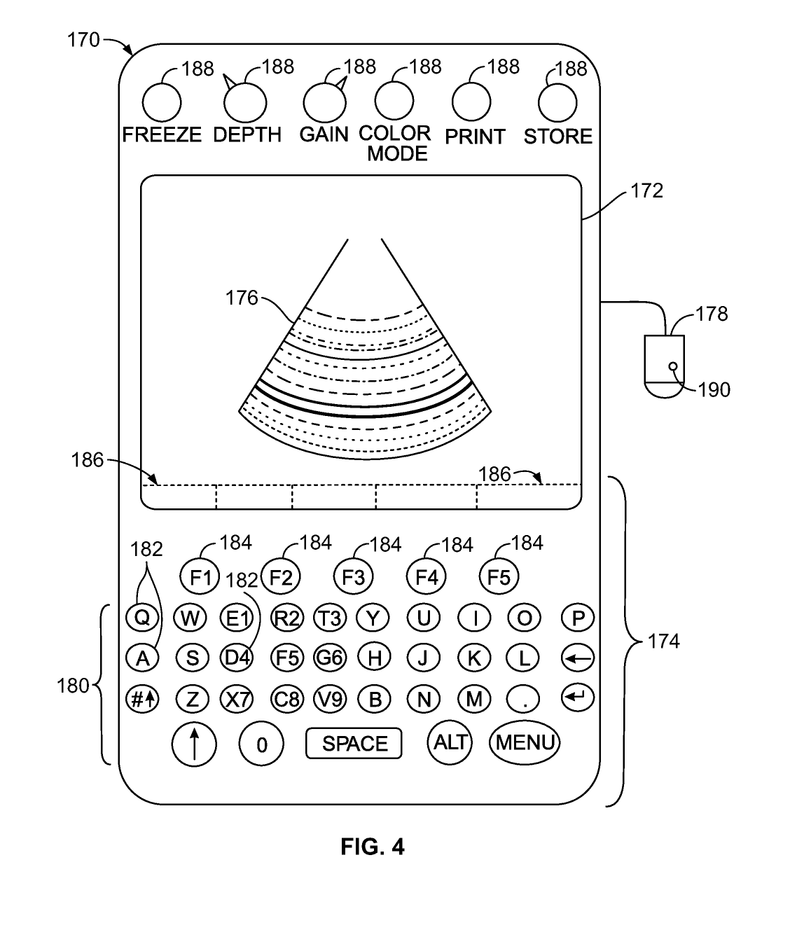

FIG. 4 illustrates a hand carried or pocket-sized ultrasound imaging system 170 wherein display 172 and user interface 174 form a single unit. By way of example, the pocket-sized ultrasound imaging system 170 may be approximately 2 inches wide, approximately 4 inches in length, and approximately 0.5 inches in depth and weighs less than 3 ounces. The display 172 may be, for example, a 320×320 pixel color LCD display (on which a medical image 176 may be displayed). A typewriter-like keyboard 180 of buttons 182 may optionally be included in the user interface 174. A probe 178 is interconnected with the system 170 and may have an LED 190 located under an outer casing as discussed herein.

Multi-function controls 184 may each be assigned functions in accordance with the mode of system operation. Therefore, each of the multi-function controls 184 may be configured to provide a plurality of different actions. Label display areas 186 associated with the multi-function controls 184 may be included as necessary on the display 172. The system 170 may also have additional keys and/or controls 188 for special purpose functions, which may include, but are not limited to “freeze,” “depth control,” “gain control,” “color-mode,” “print,” and “store.”

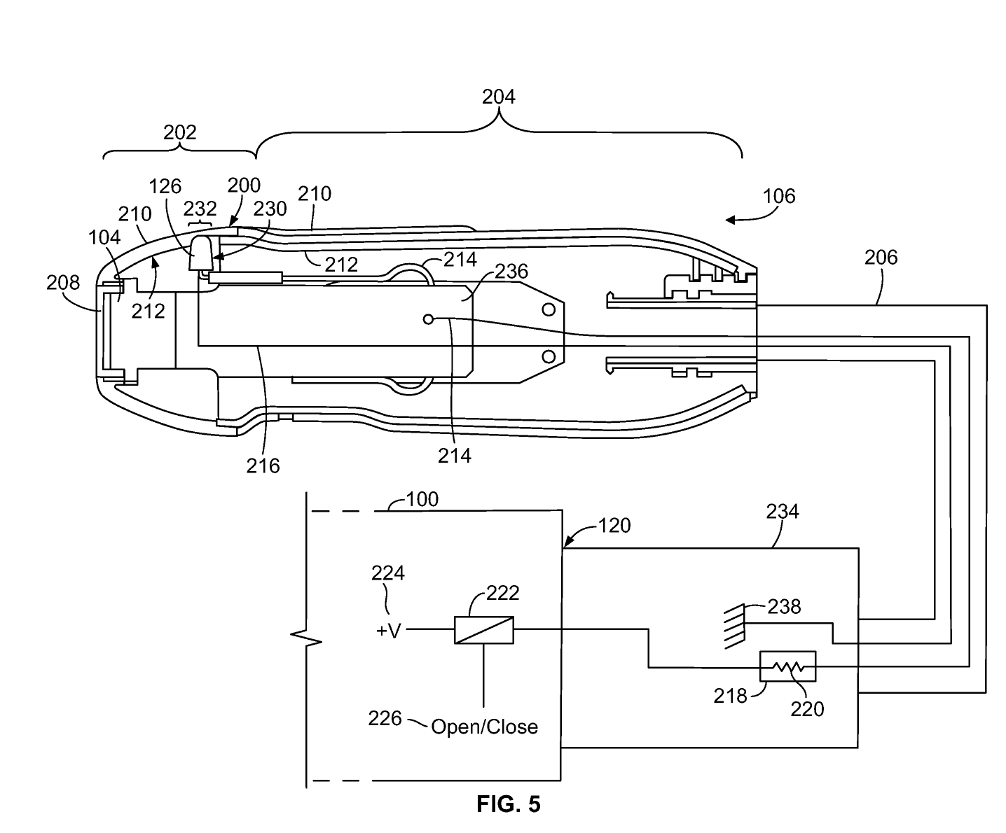

FIG. 5 illustrates an exemplary cross-sectional view of the probe 106 having the LED 126 located beneath an outer cover or casing 200. The probe 106 may be generally described as having a scan head 202, a handle 204, a cable 206 and a connector 234 that connects to the probe port 120. In some embodiments, the probe 106 may be directly connected to the system 100, and thus may not have a separate connector 234. In these cases, components and functionality housed within the connector 234 as discussed below may be provided within the system 100 or elsewhere within the probe 106. The transducer elements 104 are located in the scan head 202. A lens 208 is formed over the elements 104 and is the portion of the probe 106 that is placed on a patient when scanning. The cable 206 conveys signals between the probe 106 and the probe port 120 or system 100, such as through coaxial wires (not shown).

The casing 200 has an outer surface 210 and an inner surface 212 that encases the probe 106, preventing contaminants such as liquid and dust from entering the enclosure. The outer surface 210 is smooth, formed without holes, grooves or other asperities that may trap contamination. The casing 200 may be formed of one or more layers such as plastic, composite, rubber, silicon or other materials or combinations of materials. In some embodiments, a paint layer (not shown) may be applied to the outer surface 210 and in other embodiments, the plastic or other material may be colored, imprinted, or otherwise provided with a desired color, graphics and the like.

The LED 126 is positioned proximate the inner surface 212 of the casing 200 and is mounted and contained entirely within the casing 200. Although shown within the scan head 202 portion of the probe 106, it should be understood that the LED 126 may be located along the inner surface 212 within the handle 204. The casing 200 may be formed, such as through a plastic molding process or plastic injection process, to have an LED locating portion 232 that protrudes from and is integral with the casing 200. The LED locating portion 232 holds and/or facilitates the positioning of the LED 126 in a predetermined position. For example, the LED 126 may be angled to shine light through one particular side of the probe 106 or scan head 202, such as to indicate a predetermined orientation of the probe 106 with respect to the transducer elements 104 or an indication on the outer surface 210 of the probe 106. Although the LED 126 is shown as positioned approximately parallel to the lens 208, it should be understood that the LED 126 may be positioned in other directions, such as transverse to the lens 208.

In one embodiment, the LED 126 may be fixed to the LED locating portion 232 using glue or other adhesive, or the LED 126 may be held by the LED locating portion 232 with a pressure or force fit. In another embodiment, the inner surface 212 of the casing 200 may be provided without the LED locating portion 232 and the LED 126 may be fixed directly to a desired location of the inner surface 212 with glue or other adhesive. In yet another embodiment, the LED 126 may be held in place by surrounding structures, such as held by force between the inner surface 212 of the casing 200 and the wires, components, circuit boards and/or other structures (not shown) within the probe 106.

The LED 126 may be provided with two leads. One positive lead 214 may be configured to receive a level of power while a negative lead 216 may be connected to ground 238. The leads 214 and 216 are shown as connecting to a circuit board 236 within the handle 204. The leads 214 and 216 have corresponding wiring extending through the cable 206 to a circuit board 218 inside the connector 234 that is connected to the probe port 120 and/or directly to the system 100. By way of example only, the lead 214 may be connected to a voltage power source 224 (delivered by the system 100) through a resistor 220. Although a single resistor 220 is illustrated, it should be understood that the resistor 220 represents a resistance circuit and that other components and/or combinations of components may be used. It should be understood that the resistor 220 may be located in other areas than as shown, such as within the system 100 or the handle 204. The resistor 220 enables providing characteristic current required by the LED. In one embodiment, the power source 224 may provide positive five volts, although other voltage levels may be used with suitable resistor values. Therefore, the power source 224 and the resistor 220 may be capable of providing characteristic levels of voltage and current required for the LED functioning. In one embodiment, characteristic levels of voltage and current may be 3.7 volts and 20 milliamps, respectively. In other embodiments, other LEDs may require different characteristic levels of voltage and current.

When the probe 106 is selected or activated, the LED 126 illuminates, indicating that the probe 106 is active. The illumination from the LED 126 is visible from outside the casing 200. The selection of the probe 106 is accomplished by the system 100 when an operator makes a selection or a particular action is performed. For example, the operator may make a selection on the user interface 124, the probe 106 may be automatically selected based on a protocol that is running, or the probe 106 may be selected when the probe 106 is picked up or held by the operator. In another example, a switch (not shown) may be activated when the probe 106 is lifted from a probe holder (not shown) on the system 100, or the probe 106 may be capable of detecting an operator's touch.

When the probe 106 is selected, system 100 provides voltage power through a probe signal to the LED 126. For example, an open or close signal 226 may be sent to a switch 222, such as by the processor module 116, allowing the LED 126 to be not powered or powered, respectively. The resistance value of the resistor 220 may be defined based on the level of the voltage power source 224 and the requirements for the functioning of the LED 126. Resistance, determined from voltage power and characteristic voltage of the LED, is calculated to provide level of characteristic current for the LED. For example, for 5V power source and an LED with 3.7V characteristic voltage (and 20 mA characteristic current), the voltage at resistor terminals is 2.3V. In order to provide 20 mA current for the LED, the resistance in the circuit should be 65Ω. Including wire resistance of about 30Ω, resistance of the resistor 220 is 35Ω. When the probe 106 is not selected and the probe signal delivered by the system 100 shuts down, which in one embodiment may be the open signal 226 causing the switch 222 to open, power is removed from the LED 126. The LED 126 is therefore not illuminated when the probe 106 is not selected. It should be understood that other components and selection mechanisms may be used to provide and remove power to and from the LED 126.

In one embodiment, the LED 126 may include a semiconductor die (not shown) that emits light when power is applied. The light may be a white or colored light. A lens 230 forms a domed cover over the die, although other shapes are contemplated. The lens 230 may be clear or may be a color such as blue, green, red, yellow and the like.

The brightness, also referred to as intensity or luminance, of LEDs may be measured in millicandela (mcd). The LED 126 that is mounted proximate the inner surface 212 of the casing 200 may be an ultra-bright LED. For example, the LED 126 may have an intensity of about 10,000 mcd. To achieve this level of intensity, the LED 126 may need about four volts of forward voltage and about twenty mA of forward current, which are provided due to the resistance value of the resistor 220. In another embodiment, an LED 126 that provides less luminance than an ultra-bright LED may be used, as long as the luminance is powerful enough to shine through the casing 200. For example, a lower intensity LED 126 may have an intensity of about one mcd. In this case, assuming that the power source 224 is the same, the resistance value of the resistor 220 may be increased relative to the resistance value used with the ultra-bright LED, such as to provide about two volts of forward voltage and about ten mA of forward current. Therefore, LEDs 126 having different intensities may be mounted within the casing 200 and the desired voltage and current values may be achieved by modifying the resistance value of the resistor 220.

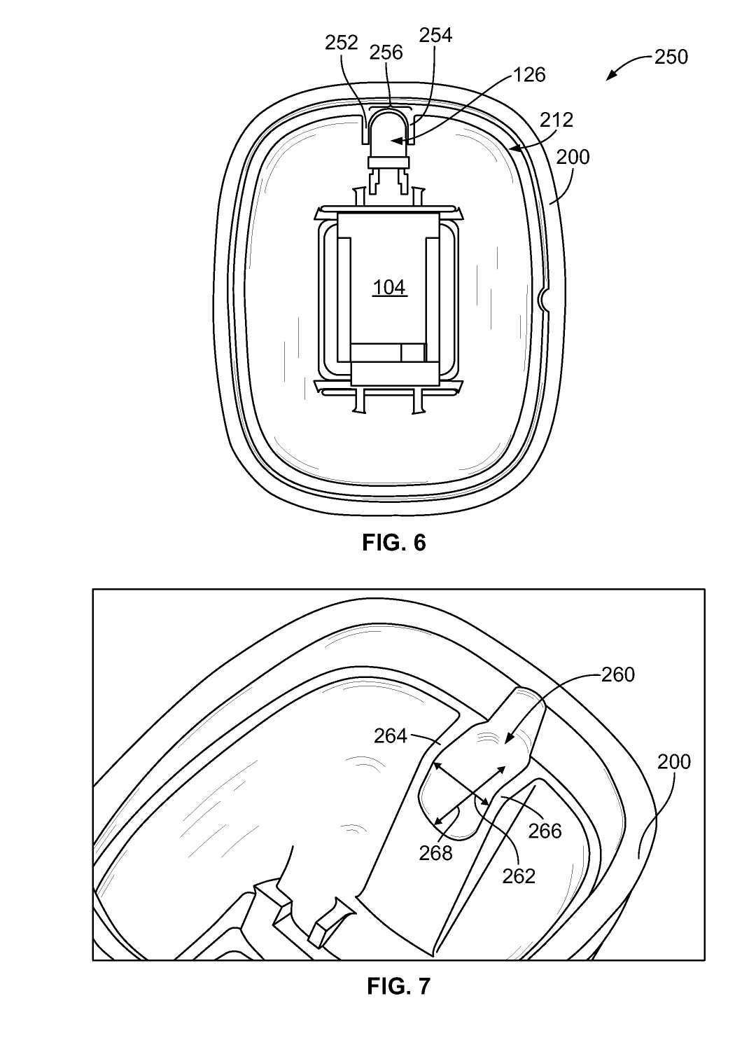

FIG. 6 illustrates a cut-away view 250 of the casing 200 and LED 126 as viewed through the lens 208 of the scan head 202. The casing 200 may be formed through injection molding or other manufacturing process. The LED locating portion 232 of FIG. 5 may be formed of one or more protrusions 252 and 254 that extend from the inner surface 212 of the casing 200. In one embodiment, two protrusions 252 and 254 may extend to form a C-shaped cavity 256 having a shape and size configured to hold the LED 126 between the protrusions 252 and 254. For example, the protrusions 252 and 254 may extend away from the inner surface 212 of the casing 200 approximately half the height of the lens 230 of the LED 126. The LED 126 may be glued or otherwise fixed to or interconnected with the protrusions 252 and 254. In another embodiment, the inner surface 212 may be formed with only one protrusion 252 or 254, and the LED 126 may be glued to at least the protrusion 252 or 254.

FIG. 7 illustrates an embodiment wherein a cup-shaped cavity 260 is formed integral with the inner surface 212 of the casing 200. For example, the cup-shaped cavity 260 may be formed during a plastic injection molding process. The cup-shaped cavity 260 has a substantially curved shape that has a width 262 between protrusions 264 and 266 that may be selected based on the size of the LED 126. A depth 268 of the cavity 260 may also be based on the size of the LED 126. In one embodiment, a three millimeter radial LED may be used, and thus the width 262 and depth 268 may be determined to accommodate the LED and a small amount of glue or adhesive. The LED 126 may be positioned and glued within the cup-shaped cavity 260 in the orientation shown in FIGS. 5 and 6.

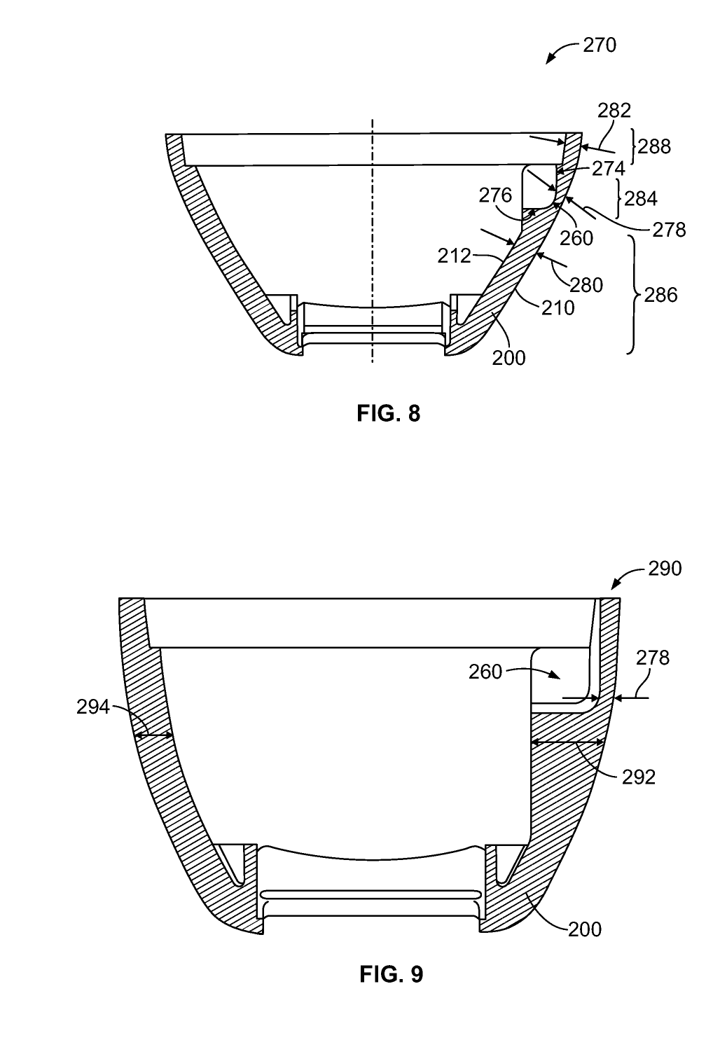

FIG. 8 illustrates a cut-away view 270 of the casing 200 as viewed transverse to the lens 208 of the scan head 202. The cup-shaped cavity 260 is formed in the inner surface 212 of the casing 200. The cup-shaped cavity 260 may have an outer side wall 274 and a bottom wall 276. Although not indicated, side walls are also formed, such as by the protrusions 264 and 266 as shown in FIG. 7. In contrast, the C-shaped cavity 256 shown in FIG. 6 may not have a bottom wall.

In one embodiment, a thickness 278 of the casing 200 may be thinner proximate to the cavity 260, such as within region 284, than in other areas of the casing 200 away from the LED 126, such as thicknesses 280 and 282 in regions 286 and 288, respectively, to further facilitate the light penetrating through the casing 200. In another embodiment, the thickness 278 of the casing 200 at least proximate the LED 126 may be adjusted to increase the amount of light penetrating through the casing 200 without having to increase the luminance of the LED 126. In yet another embodiment, different materials having different levels of translucence may be used from the casing to increase the amount of light penetrating through the casing 200 from the LED 126. In still another embodiment, the pigment, color or paint of the material forming the casing 200 at least proximate the LED 126 may be adjusted to allow more light from the LED 126 to penetrate through the casing 200.

FIG. 9 illustrates another cut-away view 290 that shows the cup-shaped cavity 260 formed in the casing 200. In this embodiment, thickness 292 of the casing 200 below the cavity 260 is thicker than the thickness 278 proximate the cavity 260 as well as a thickness 294 on an opposite side of the casing 200. In one embodiment, the casing 200 may be thicker below the cavity 260 for ease of manufacturing. It should be understood that the thickness and shape of the casing 200 may vary depending on the type and style of the probe 106.

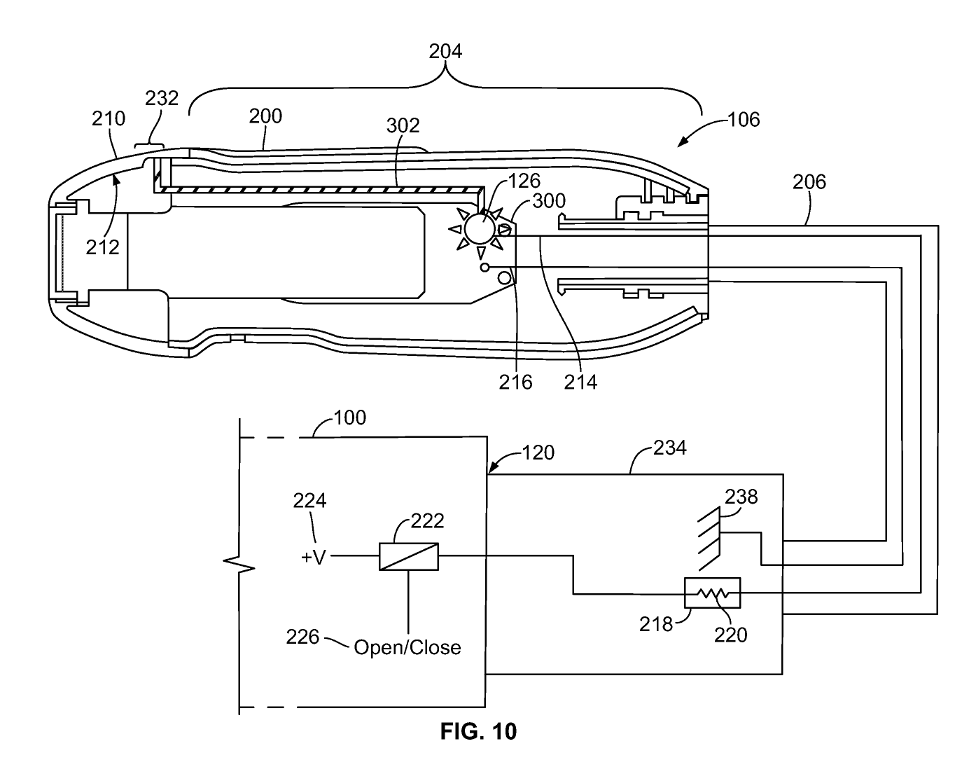

FIG. 10 illustrates another embodiment wherein the LED 126 is located beneath the casing 200. The LED 126 is positioned away from the inner surface 212 of the casing 200. As shown, the LED 126 is located within the handle 204 although other positions are contemplated. The LED 126 may be held in a desired position by a circuit board 300 or other structure. A light guide 302, such as a fiber optic, may conduct light from the LED 126 to the inner surface 212 of the casing 200. Other materials and structure may be used to guide the light between the LED 126 and the casing 200. The light guide 302 may be positioned to shine the light through the casing 200 to indicate the orientation of the probe 106. For example, the light guide 302 may be positioned within the locating portion 232. If needed, the light guide 302 may be held in position with glue, adhesive, or other fasteners. In some embodiments, the light guide 302 may be used together with the LED 126 that is positioned away from the inner surface 212 to minimize thermal heating of the probe casing 200 that may result from contact between the LED 126 and the casing 200.

A technical effect of at least one embodiment is the ability to form a smooth outer surface of the casing that encloses an ultrasound probe. The LED that indicates when the probe is active and that may also indicate the orientation of the probe is mounted beneath, and shines through, the casing. No hole is provided within the casing for the LED to protrude through, and thus the cleaning and disinfecting of the probe is easier and faster for the operator. In some embodiments, an ultra-bright LED may be used to generate sufficient luminosity to penetrate through the casing. The inner surface of the casing may also be provided with an LED locator portion, such as a cavity or protrusion(s), to facilitate positioning and holding the LED in a desired location and/or orientation.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, values and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the fill scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An ultrasound probe, comprising:
a probe casing comprising an inner surface and an outer surface; and
a light emitting diode (LED) mounted and contained entirely within the probe casing, the LED configured to be illuminated when the probe is active.

2. The ultrasound probe of claim 1, wherein the inner surface of the probe casing comprises one of a C-shaped cavity and a cup-shaped cavity configured to hold at least a portion of the LED.

3. The ultrasound probe of claim 1, wherein the inner surface of the probe casing comprises at least one protrusion configured to hold at least a portion of the LED, and wherein the LED being interconnected with the protrusion with at least one of glue and an adhesive.

4. The ultrasound probe of claim 1, wherein the probe further comprises a scan head, a handle and a cable, and wherein the LED is located within one of the scan head and the handle.

5. The ultrasound probe of claim 1, wherein the LED comprises an ultra-bright LED.

6. The ultrasound probe of claim 1, wherein the LED has a luminance of approximately 10,000 millicandela.

7. The ultrasound probe of claim 1, wherein the LED extending from the inner surface to be inside the probe casing.

8. The ultrasound probe of claim 1, wherein the LED is interconnected to the inner surface with at least one of glue and an adhesive.

9. The ultrasound probe of claim 1, further comprising a light guide configured to conduct light from the LED to the inner surface of the probe casing.

10. A method for indicating whether an ultrasound probe is active, the method comprising:
locating a light emitting diode (LED) beneath a probe casing, the probe casing configured to enclose at least the LED and an array of transducer elements; and
providing power to the LED when the probe is selected, the LED configured to provide a level of luminescence that is visible outside the probe casing.

11. The method of claim 10, wherein an inner surface of the probe casing is configured to provide an LED locating portion, the method further comprising attaching the LED to the LED locating portion.

12. The method of claim 10, wherein the inner surface of the probe casing is configured to provide at least one of a C-shaped cavity, a cup-shaped cavity and at least one protrusion, the method further comprising fixing the LED to the at least one of the C-shaped cavity, the cup-shaped cavity and the at least one protrusion by at least one of pressure, glue, and an adhesive.

13. The method of claim 10, wherein the probe casing comprises an inner surface and an outer surface, the method further comprising forming the outer surface of the probe casing without holes, grooves and asperities proximate the LED.

14. The method of claim 10, wherein the LED comprises a positive lead configured to be connected to a power source and a negative lead configured to be connected to ground, the method further comprising:
positioning a resistor between the power source and the positive lead; and
selecting a value for the resistor based on at least a luminance value of the LED.

15. The method of claim 10, the method further comprising positioning the LED beneath the probe casing to indicate an orientation of the probe with respect to the transducer elements.

16. An ultrasound probe, comprising:
a probe casing comprising an inner surface and an outer surface, the probe casing having a thickness; and
a light emitting diode (LED) mounted within the probe casing and positioned to indicate an orientation of the probe, the thickness of the probe casing in a region proximate the LED being thinner than in areas of the probe casing located away from the LED, the LED configured to have a luminescence that is visible outside the outer surface when the LED is activated.

17. The ultrasound probe of claim 16, further comprising an LED locating portion that protrudes from the inner surface of the probe casing, the LED being mounted proximate the LED locating portion.

18. The ultrasound probe of claim 16, further comprising a cup-shaped cavity formed integral with the inner surface of the probe casing, the cup-shaped cavity comprising a width and a depth based on a size of the LED, and wherein the LED is fixed to the cup-shaped portion by at least one of pressure, glue and an adhesive.

19. The ultrasound probe of claim 16, wherein the LED is located in a scan head portion of the probe and oriented to shine light out of one side of the scan head portion through the probe casing.

20. The ultrasound probe of claim 16, wherein the luminance of the LED is one of approximately 10,000 millicandela and less than 10,000 millicandela.
